(19) United States
(12) Patent Application Publication
(10) Pub. No.: US 2025/0319468 A1
(43) Pub. Date: Oct. 16, 2025

(54) DEVICE FOR CAPTURING A BIOLOGICAL OBJECT, AND METHOD AND SYSTEM FOR ANALYSING A BIOLOGICAL OBJECT USING THE CAPTURING DEVICE

(71) Applicant: COMMISSARIAT A L'ENERGIE ATOMIQUE ET AUX ENERGIES ALTERNATIVES, Paris (FR)

(72) Inventor: Vincent AGACHE, Grenoble cedex 09 (FR)

(73) Assignee: COMMISSARIAT A L'ENERGIE ATOMIQUE ET AUX ENERGIES ALTERNATIVES, Paris (FR)

(21) Appl. No.: 18/866,674

(22) PCT Filed: May 22, 2023
(86) PCT No.: PCT/EP2023/063555

§ 371 Date: Nov. 18, 2024

(30) Foreign Application Priority Data
May 25, 2022 (FR) FR2205064

(51) Int. Cl. B01L 3/00

(52) U.S. Cl. CPC B01L 3/502761; B01L 2200/026; B01L 2200/0668; B01L 2200/12; B01L 2300/0663

(57) ABSTRACT

A device for capturing a biological object includes a first fluid circuit. The first fluid circuit includes: a first fluid port and a second fluid port; a main channel extending between the first fluid port and the second fluid port, including a first branch into which the first fluid port opens, a central branch forming an extension of the first branch via a first junction branch, and a second branch forming an extension of the central branch via a second junction branch and opening at the second fluid outlet; and a first hydrodynamic trap and a second hydrodynamic trap, each hydrodynamic trap taking the form of a branch from the main channel and including a housing sized to accept the biological object and a restriction forming an extension of the housing.

### TECHNICAL FIELD OF THE INVENTION

The invention relates to a device for capturing a biological object, and a method and a system for analyzing a biological object using said capturing device.

### PRIOR ART

It is known to wish to isolate a biological object in a fluid circuit to study its secretions and its reactions to various substances. This is the case for example for tumor cells that will be made to secrete extracellular vesicles, such as exosomes. To isolate the biological object it is known to use a hydrodynamic trap in which the biological object becomes trapped throughout the duration of the analysis. This type of hydrodynamic trap is described in particular in the following publications:
Wei-Heong Tan and Shoji Takeuchi, A trap-and-release integrated microfluidic system for dynamic microarray applications, Jan. 23, 2007, 104(4) 1146-1151, PNAS.
R J Kimmerling, G L Szeto, J W Li, A S Genshaft, S W Kazer, K P Payer, J D Borrajo, J C Blainey, D J Irvine, A K Shalek, S R Manalis. A microfluidic platform enabling single cell RNA-seq of multigenerational lineages, Nature Communications, 7, Article number: 10220. (2016).

However, depending on the manipulations effected, it is common to create pressure differences in the fluid circuit liable to reverse the direction of flow in the fluid circuit. This can occur if the operator is called upon to change the media to be injected into the fluid circuit or simply if the pressures are out of balance between the various fluid ports. In this situation the biological object may be caused to escape from its hydrodynamic trap, thus disturbing the analysis.

The patent application US2021/039104A1 describes a cell trapping system using a plurality of traps. It enables the cells to be trapped when they are injected into the fluid circuit in one direction or the opposite direction. However, its structure does not enable a biological object to be retained in its fluid circuit in the event of reversal of the direction of flow in the fluid circuit.

The aim of the invention is to propose a solution for maintaining the biological object in the fluid circuit even in the event of reversal of the direction of flow in the fluid circuit.

### SUMMARY OF THE INVENTION

The above aim is achieved by a device for capturing a biological object including a first fluid circuit, said first fluid circuit having:
a first fluid port and a second fluid port,
a main channel extending between the first fluid port and the second fluid port, comprising a first branch into which the first fluid port opens, a central branch forming an extension of the first branch via a first junction branch, and a second branch forming an extension of said central branch via a second junction branch and opening out at the second fluid outlet,
a first hydrodynamic trap and a second hydrodynamic trap,
each hydrodynamic trap taking the form of a branch from the main channel and including a housing sized to accept said biological object and a restriction forming an extension of said housing,
the first branch and the second branch of the main channel having no housing that is part of a hydrodynamic trap,
the first hydrodynamic trap being arranged so that its housing communicates on one side with the central branch of the main channel and its restriction opens on the other side into the first branch of the main channel, and
the housing of the second hydrodynamic trap communicates on one side with the central branch of the main channel and its restriction opens on the other side into the second branch of the main channel.

In contrast to the teaching of the patent application US2021/039104A1, note that the capture device of the invention has no hydrodynamic trap in the first branch or the second branch of its main channel. In the device described in the above prior art document these two branches include housings for capturing the cells, which does not enable a device of this kind to meet the objective of retaining the biological object in the fluid circuit. In fact, when the cells are injected into the circuit in a first direction of flow (“forward flow” for example in document D1) they come to be lodged in the first housing accessible in the first branch. In the event of reversal of the direction of flow (“reverse flow”) the cells are able to escape from these first housings and therefore are not retained in the fluid circuit. The structural difference between the invention and the prior art device therefore makes it possible to obtain the technical effect of retaining the biological object in the fluid circuit even in the event of reversing the flow.

The invention is also directed to an analysis system using said capture device, the capture device guaranteeing retention of the biological object in the fluid circuit throughout the analysis, even in the event of reversal of the direction of flow in the fluid circuit.

The system for analysis of a biological object includes a device for capturing said biological object and a measuring device:
the capture device being as defined hereinabove,
the measuring device including:
   a measurement fluid branch connected at a first end to the central branch of the capture device between its two hydrodynamic traps and at a second end to a second fluid circuit distinct from the first fluid circuit, and
   a sensor cooperating with the measurement fluid branch.

In accordance with one feature the second fluid circuit includes a recovery fluid branch extending between a third fluid port and a fourth fluid port of the second fluid circuit, said second end of the measurement fluid branch being connected to the recovery fluid branch between the third fluid port and the fourth fluid port.

In accordance with another feature the sensor is of resonator type, of photonic type, or configured to measure an electrical parameter in the measurement fluid branch.

In accordance with another feature the first fluid circuit and the second fluid circuit are produced in a component manufactured by micro-manufacture.

The invention is finally aimed at an analysis method implemented with the aid of said analysis system.

The method for analysis of a biological object is implemented with the aid of the analysis system as defined hereinabove, the method including:
a first step of injection of the biological object via the first fluid port by application of a first pressure at the first fluid port greater than a second pressure at the second fluid port,
hydrodynamic trapping of the biological object in the second hydrodynamic trap,
application of a third pressure to the first fluid port and the second fluid port and application of a fourth pressure to the third fluid port greater than a fifth pressure at the fourth fluid port and less than said third pressure,
recovery of the secretions generated by said biological object at the fourth fluid port after passage through the measurement fluid branch.

### BRIEF DESCRIPTION OF THE FIGURES

Other features and advantages will become apparent in the following detailed description with reference to the appended drawings, in which:

FIG. 1 represents a device in accordance with the invention for capturing a biological object.

FIGS. 2A to 2E depict the operating principle of the capture device from FIG. 1.

FIG. 3 represents a system according to the invention for analysis of a biological object incorporating said capture device from FIG. 1.

FIGS. 4A to 4C depict the various steps of a method according to the invention of analyzing a biological object.

### DETAILED DESCRIPTION OF AT LEAST ONE EMBODIMENT

In the remainder of the description by fluid branch is meant a single fluid channel with only two ends and in which a fluid can circulate, each end forming a distinct fluid port providing access to said channel.

In the remainder of the description by biological object is meant for example a cell, an aggregate of cells, a virus, a bacterium or other biological object. By aggregate of cells is meant the self-assembly of one or more types of cells in three dimensions. An aggregate of cells of this kind can in particular be termed a spheroid, an organoid, a neuro-sphere. Without this being limiting on the invention, the biological object may for example have a diameter from around one hundred nm to a few μm.

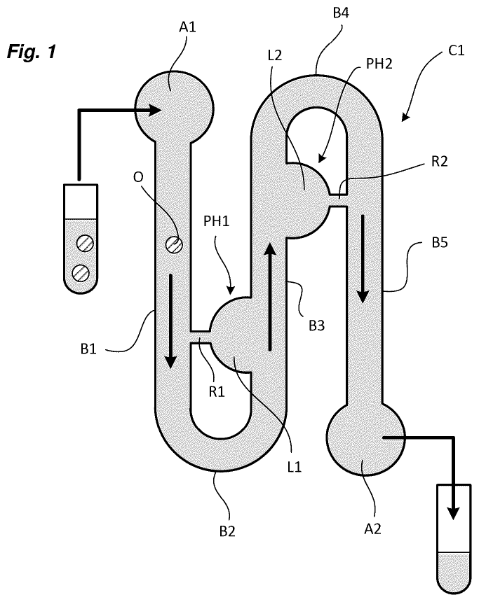

FIG. 1 represents a device for capturing a biological object O.

This device includes in particular a fluid network advantageously implemented in a fluid component. The fluid network includes fluid branches formed of channels integrated into said component. These channels may be produced by machining for example or with the aid of any other method such as micro-manufacture. The component may be based on glass and/or silicon.

In more concrete terms, the capture device includes a first fluid circuit C1.

The first fluid circuit C1 includes a first fluid port A1 and a second fluid port A2. By fluid port is meant a fluid inlet or outlet through which it is possible to inject a fluid or to recover a fluid. Each fluid port may have the fluid inlet or fluid outlet role, depending on the direction of flow of the fluid in the fluid circuit.

The first fluid circuit C1 includes a main channel extending between the first fluid port A1 and the second fluid port A2.

Without this being limiting on the invention, the main channel includes a first branch B1 into which the first fluid port A1 opens, a central branch B3 extending said first branch B1 via a first junction branch B2, and a second branch B5 extending said central branch B3 via a second junction branch B4 and opening into the second fluid port A2.

By way of non-limiting example, the first branch B1, the central branch B3 and the second branch B5 have a rectilinear shape. Of course, they could have some other shape.

Likewise, by way of non-limiting example the first junction branch B2 and the second junction branch B4 both have a bent shape, enabling a junction to be made respectively between the first branch B1 and the central branch B3 on the one hand and between the central branch B3 and the second branch B5 on the other hand.

The first fluid port A1, the first branch B1, the first junction branch B2, the central branch B3, the second junction branch B4, the second branch B5 and the second fluid port A2 are sized to enable the biological object O to circulate and to move in the first fluid circuit C1 when it is placed in a vector fluid.

The first fluid circuit C1 also includes at least a first hydrodynamic trap PH1 and a second hydrodynamic trap PH2.

In the context of the invention a hydrodynamic trap is intended to be able to trap the biological object O in such a manner as to enable better surveillance of its secretions and its behavior, for example if it is subjected to treatment by various substances.

Each hydrodynamic trap PH1, PH2 takes the form of a branch from the main channel.

Thus a hydrodynamic trap includes a housing L1, L2 sized to receive said biological object O and a restriction R1, R2 extending said housing in such a manner as to form a sort of funnel.

According to the invention the two hydrodynamic traps PH1, PH2 of the device are arranged mirror fashion. In other words, the first hydrodynamic trap PH1 is arranged so that its housing L1 communicates on one side with the central branch B3 of the main channel and its restriction R1 opens on the other side into the first branch B1 of the main channel.

Conversely, the second hydrodynamic trap PH2 is arranged so that its housing L2 communicates on one side with the central branch B3 of the main channel and its restriction R2 opens into the second branch B5 of the main channel.

Thanks to this configuration the biological object O is always retained in one of the two traps, whatever the direction of flow of the fluid in the first fluid circuit C1.

It should be noted that the first branch B1 and the second branch B5 are free of housings for receiving a biological object O. In other words, the biological object can be trapped only when it is situated in the central branch B3 of the main channel, in one or the other of the housings L1, L2 of the two hydrodynamic traps PH1, PH2. This particular structural feature enables the biological object O to be retained in the central branch B3 of the main channel even in the event of reversal of the direction of flow.

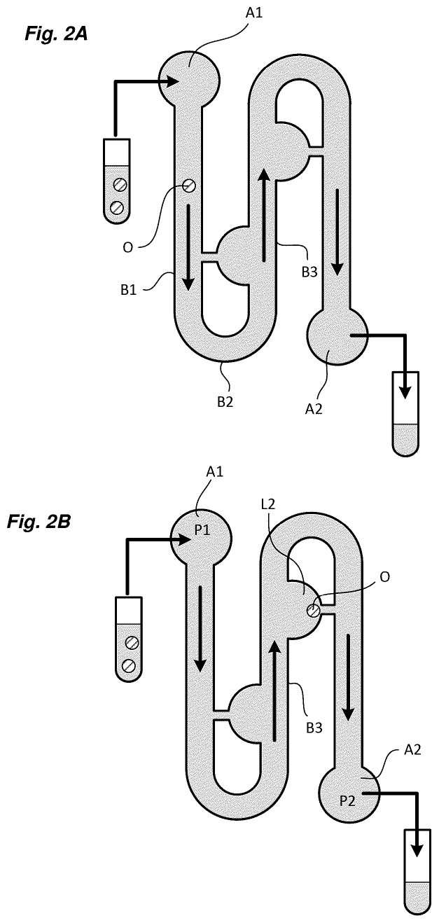

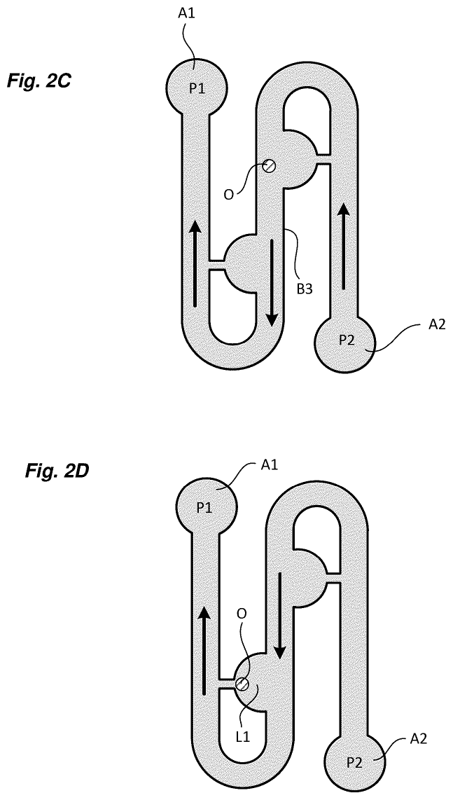

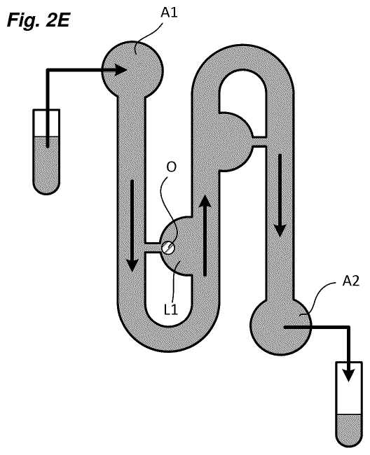

This principle is explained below with reference to FIGS. 2A to 2E.

FIG. 2A: A vector fluid containing the biological object O is injected through the first fluid port A1.

The vector fluid therefore passes through the first branch B1 and then the first junction branch B2 so that the biological object O is able to reach the central branch B3.

FIG. 2B: Given the pressure difference (P1>P2) between the first fluid port A1 and the second fluid port A2, the biological object O cannot be captured in the first hydrodynamic trap PH1. The biological object O then encounters the housing L2 of the second hydrodynamic trap PH2 communicating with the central branch B3 and comes to be lodged therein.

FIG. 2C: Thereafter, when the direction of flow in this first fluid circuit is reversed (for example in the event of a pressure drop between the first fluid port and the second fluid port (P2>P1), the biological object O is dislodged from the second hydrodynamic trap PH2 and returns to the central branch B3.

FIG. 2D: As the two hydrodynamic traps PH1, PH2 are arranged mirror fashion the biological object O is directed toward the first fluid port A1 and encounters on its way the housing L1 of the first hydrodynamic trap PH1 and comes to be lodged therein.

FIG. 2E: After the medium in the first fluid circuit is changed the biological object O remains trapped in the housing L1 of the first hydrodynamic trap PH1.

Thus if the direction of flow in the circuit is reversed the biological object O leaves the hydrodynamic trap in which it is located and comes to be lodged in the symmetrical trap, that is to say always the trap disposed at the greater downstream distance in the direction of flow of the fluid in the circuit.

This geometry and the mirror configuration of the two hydrodynamic traps PH1, PH2 therefore makes it possible to guarantee that the biological object O is trapped in the fluid circuit at all times, independently of the residual pressure gradient and connections and disconnections at the level of the first fluid port A1 and the second fluid port A2.

This capture device and its operating principle are particularly useful in a system for analysis of a biological object.

The analysis could consist for example in study of the secretions of a cell or an organoid. The secretions may for example include exosomes, extracellular vesicles, viral particles, proteins, circulating RNA type molecules, circulating DNA, . . .

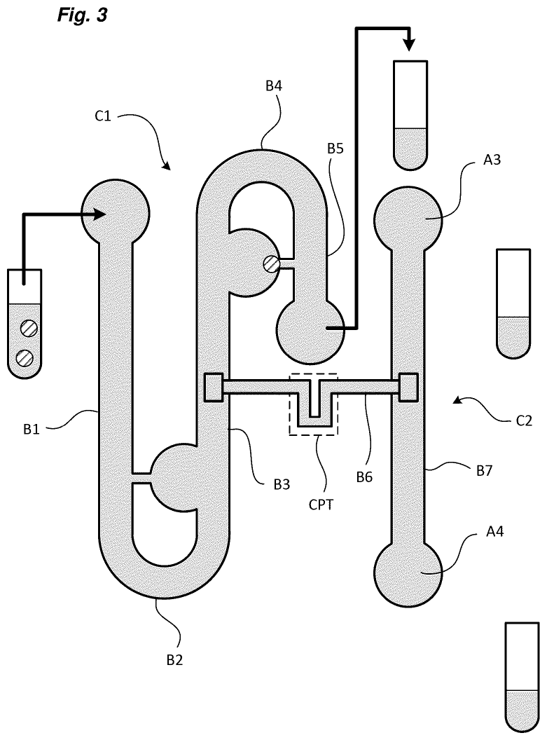

Referring to FIG. 3, this analysis system therefore includes a device as described hereinabove for capturing the biological object O and a measuring device.

The measuring device includes a measurement fluid branch B6 having a first end connected to the central branch B3 of the first fluid circuit C1 between the two hydrodynamic traps PH1, PH2 and a second end connected to a second fluid circuit C2.

The measurement fluid branch B6 carries a sensor CPT configured to carry out measurements on the fluid passing through the measurement fluid branch. The sensor CPT may for example be positioned on the measurement fluid branch B6 or have the latter pass through it. Of course, this will depend on the type of sensor employed. It may in particular be a resonator type sensor, more particularly known as a “Suspended Nanochannel Resonator” (SNR), that is positioned to have the measurement fluid branch B6 pass through it. Such an SNR type sensor is described in particular in the publication entitled:
“Suspended Nanochannel Resonator Arrays with Piezoresistive Sensors for High-Throughput Weighing of Nanoparticles in Solution”-Marco Gagino et al.-ACS Sens 2020, 5, 1230-1238.

Of course, it would be possible to use other types of sensor. One solution would be for example to measure an electrical parameter (resistance/electric current) across a particle that has come to be trapped in a micropore/nanopore or passed through it in a transitional manner, or generally speaking a fluid restriction. Another example would be to use an optical interferometer (Mach-Zehnder) type photonic sensor having a surface functionalized as a function of targets secreted by the biological object O and intended to pass through the measurement fluid branch B6.

The second fluid circuit C2 advantageously enables recovery of the fluid containing the secretions generated by the biological object O following passage through the measurement fluid branch B6.

The second fluid circuit C2 therefore includes at least one recovery fluid branch B7 that extends between a third fluid port A3 and a fourth fluid port A4.

It should be noted that each fluid port of the system may be at a given pressure distinct from that of the other fluid ports.

In operation, to recover the secretions generated by a biological object O at the fourth fluid port A4 after passing through the measurement fluid branch B6 the method is as follows:

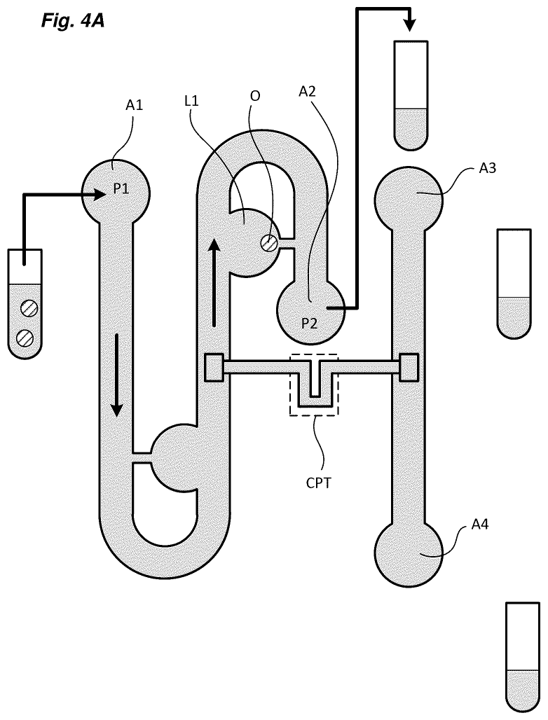

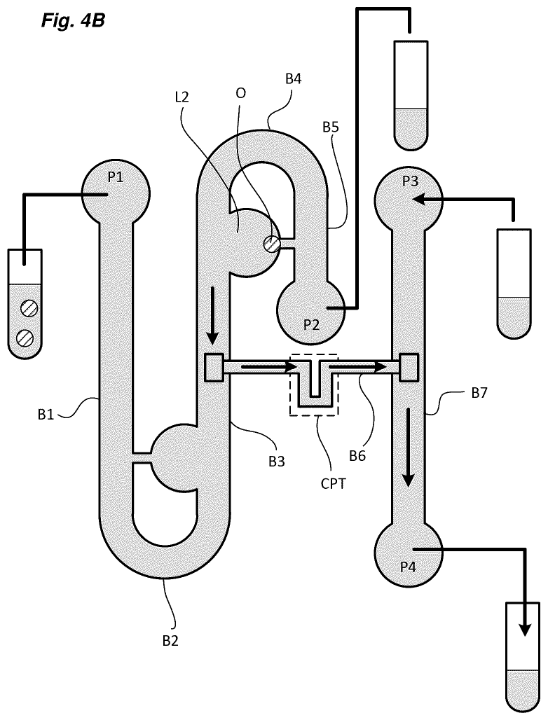

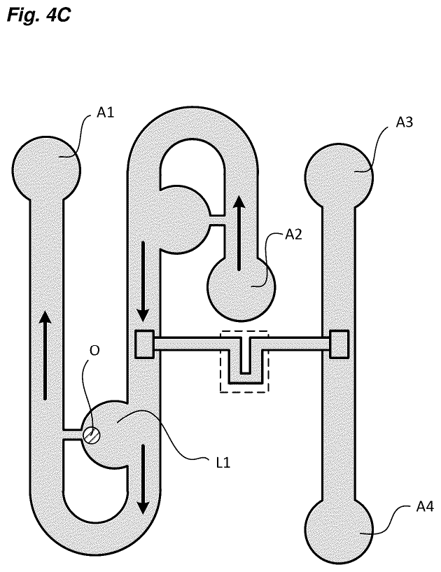

FIG. 4A: After injection via the first fluid port (P1>P2) the biological object is trapped in the second hydrodynamic trap PH2 of the capture device in accordance with the principle described hereinafter.

FIG. 4B: The first fluid port A1 and the second fluid port A2 are brought to the same pressure value P1=P2. The third fluid port A3 is placed at a pressure value higher than that present at the fourth fluid port A4: P3>P4. The pressure applied at the first fluid port A1 is also fixed and greater than that at the third fluid port A3. The secretions generated by the biological object O therefore pass through the measurement fluid branch B6 carrying the sensor CPT and are recovered in a tank connected to the fourth fluid port A4.

FIG. 4C: In the event of modification of the direction of flow in the first fluid circuit C1 (for example linked to a pressure gradient) the biological object O can be caused to become dislodged from the second hydrodynamic trap PH2 to return to the first hydrodynamic trap PH1, as described hereinabove with reference to FIGS. 2A to 2E. The analysis process may nevertheless continue on the same biological object O without loss. This principle can be applied to each fluid port, adapting the pressures exerted in the circuits.

The two fluid circuits C1, C2 can be produced in the same micro-fluid component by any appropriate technique, for example by machining and/or micro-manufacture.

The solutions described hereinabove have numerous advantages, including:
the capture device guarantees retention of a biological object in the fluid circuit even in the event of changing the direction of flow in that fluid circuit, for example following temporary disconnection of one of the fluid ports to enable a change of solution (for example a cellular culture medium or drugs, anti-viral agents, or antibiotic agents if it is a trapped bacterium) to which the biological object will be exposed;
the analysis system using a capture device of this kind enables continuous analysis of a biological object without loss (see above);
the solution is simple to design and easy to implement for the analysis of a biological object.

The invention claimed is:

1. A device for capturing a biological object, which device includes a first fluid circuit, said first fluid circuit having:
a first fluid port and a second fluid port,
a main channel extending between the first fluid port and the second fluid port, comprising a first branch into which the first fluid port opens, a central branch forming an extension of the first branch via a first junction branch, and a second branch forming an extension of said central branch via a second junction branch and opening out at the second fluid outlet,
a first hydrodynamic trap and a second hydrodynamic trap,
each hydrodynamic trap taking the form of a branch from the main channel and including a housing sized to accept said biological object and a restriction forming an extension of said housing,
wherein:
the first branch and the second branch of the main channel have no housing that is part of a hydrodynamic trap,
the first hydrodynamic trap is arranged so that its housing communicates on one side with the central branch of the main channel and its restriction opens on the other side into the first branch of the main channel, and
the housing of the second hydrodynamic trap communicates on one side with the central branch of the main channel and its restriction opens on the other side into the second branch of the main channel.

2. A system for analysis of a biological object, including a device for capturing said biological object and a measuring device, wherein:
the capture device is as defined in claim 1,
and
the measuring device includes:
a measurement fluid branch connected at a first end to the central branch of the capture device between its two hydrodynamic traps and at a second end to a second fluid circuit (C2 distinct from the first fluid circuit,
a sensor cooperating with the measurement fluid branch.

3. The system as claimed in claim 2, wherein the second fluid circuit includes a recovery fluid branch extending between a third fluid port and a fourth fluid port of the second fluid circuit, said second end of the measurement fluid branch being connected to the recovery fluid branch between the third fluid port and the fourth fluid port.

4. The analysis system as claimed in claim 2, wherein the sensor is of resonator type.

5. The system as claimed in claim 2, wherein the sensor is of photonic type.

6. The system as claimed in claim 2, wherein the sensor is configured to measure an electrical parameter in the measurement fluid branch.

7. The system as claimed in claim 2, wherein the first fluid circuit and the second fluid circuit are produced in a component manufactured by micro-manufacture.

8. method for analyzing a biological object using the analysis system as defined in claim 2, the method comprising:
a first step of injection of the biological object via the first fluid port by application of a first pressure at the first fluid port greater than a second pressure at the second fluid port,
hydrodynamic trapping of the biological object in the second hydrodynamic trap,
application of a third pressure to the first fluid port and the second fluid port and application of a fourth pressure to the third fluid port greater than a fifth pressure at the fourth fluid port and less than said third pressure,
recovery of the secretions generated by said biological object at the level of the fourth fluid port after passage through the measurement fluid branch.
